(19) United States
(12) Patent Application Publication
(10) Pub. No.: US 2025/0377281 A1
(43) Pub. Date: Dec. 11, 2025

(54) BIOLOGICAL SAMPLE ANALYSIS SYSTEM AND BIOLOGICAL SAMPLE ANALYSIS METHOD

(71) Applicant: SONY GROUP CORPORATION, Tokyo (JP)

(72) Inventor: Akio FURUKAWA, Tokyo (JP)

(21) Appl. No.: 18/874,738

(22) PCT Filed: Mar. 22, 2022
(86) PCT No.: PCT/JP2023/011266

§ 371 Date: Dec. 13, 2024

(30) Foreign Application Priority Data
Jun. 21, 2022 (JP) 2022-099396

(51) Int. Cl. G01N 15/1433; G01N 33/483

(52) U.S. Cl. CPC G01N 15/1433; G01N 33/483

(57) ABSTRACT

A biological sample analysis system (100) according to an embodiment includes a detection optical system (121), a focal point of which is set at a predetermined position in a container (C), an imaging unit (122), a light receiving surface of which is located at an image forming position of an image of light transmitted through the detection optical system (121), and an optical path length adjustment element (13) disposed on an optical path between the detection optical system (121) and the imaging unit (122) and in a part within an angle of view of the imaging unit (121).

### FIELD

The present disclosure relates to a biological sample analysis system and a biological sample analysis method.

### BACKGROUND

There has been flow cytometry as a method of analyzing (or analytically studying: in the present disclosure, it is assumed that analysis includes analytical study) proteins of biologically relevant microparticles such as cells, microorganisms, and liposomes. A device used for the flow cytometry is referred to as flow cytometer (FCM). In the flow cytometer, microparticles flowing in a channel are irradiated with laser light having a specific wavelength, light such as fluorescence, forward-scattered light, and side-scattered light emitted from the microparticles is converted into an electric signal by a photodetector and quantified, and a result of the quantification is statistically analyzed, whereby a type, sizes, structures, and the like of the individual microparticles are determined.

In recent years, a so-called imaging flow cytometer (IFCM) that acquires, with an image sensor, a two-dimensional image of fluorescence emitted from microparticles has been developed.

### CITATION LIST

### Patent Literature

Patent Literature 1: US 2004/0217256 A

### SUMMARY

### Technical Problem

However, the positions where the microparticles pass through the channel are random. For that reason, there is a problem in that the passing positions of the microparticles in the channel deviate from a focal position of an objective lens and an unfocused and unclear image is acquired.

Therefore, the present disclosure provides a biological sample analysis system and a biological sample analysis method capable of suppressing deterioration in image quality due to deviation from a focal position.

### Solution to Problem

In order to address the above mentioned problem, a biological sample analysis system according to one embodiment of the present disclosure includes: a detection optical system, a focal point of which is set at a predetermined position in a container; an imaging unit, a light receiving surface of which is located at an image forming position of an image of light transmitted through the detection optical system; and an optical path length adjustment element disposed on an optical path between the detection optical system and the imaging unit and in a part within an angle of view of the imaging unit.

### BRIEF DESCRIPTION OF DRAWINGS

FIG. 1 is a schematic diagram illustrating a configuration example of a particle analysis system according to the present disclosure.

FIG. 2 is a block diagram illustrating a more specific configuration example of the particle analysis system according to the embodiment.

FIG. 3 is a block diagram illustrating a schematic configuration example of an image sensor according to the embodiment.

FIG. 4 is a block diagram illustrating a schematic configuration example of an EVS according to the embodiment.

FIG. 5 is a diagram illustrating a schematic configuration example of a detection optical system according to a comparative example of the embodiment.

FIG. 6 is a diagram illustrating a schematic configuration example of a detection optical system according to another comparative example of the embodiment.

FIG. 7 is a diagram illustrating a schematic configuration example of a detection optical system according to the embodiment.

FIG. 8 is a diagram for explaining a principle of an optical path length adjustment element according to the embodiment.

FIG. 9 is a diagram for explaining a method of evaluating a focus state by an information processing unit according to the embodiment (part 1).

FIG. 10 is a diagram for explaining the method of evaluating the focus state by the information processing unit according to the embodiment (part 2).

FIG. 11 is a diagram for explaining a specific example of an image of a bioparticle acquired by an imaging unit according to the embodiment (part 1).

FIG. 12 is a diagram for explaining the specific example of the image of the bioparticle acquired by the imaging unit according to the embodiment (part 2).

FIG. 13 is a diagram illustrating a schematic configuration example of a detection optical system according to a first modification of the embodiment.

FIG. 14 is a diagram illustrating a schematic configuration example of a detection optical system according to a second modification of the embodiment.

FIG. 15 is a diagram illustrating a schematic configuration example of a detection optical system according to a third modification of the embodiment.

FIG. 16 is a hardware configuration diagram illustrating an example of a computer that implements functions of an information processing device according to the present disclosure.

FIG. 17 is a diagram illustrating a modification.

FIG. 18 is a diagram illustrating an example of a surface shape of an optical path length adjustment element.

FIG. 19 is a diagram illustrating another modification. Description of Embodiments

Embodiments of the present disclosure are explained in detail below with reference to the drawings. Note that, in the embodiments explained below, redundant explanation is omitted by denoting the same parts with the same reference numerals and signs.

The present disclosure is explained according to the item order explained below.
1. Schematic configuration example of a particle analysis system
2. Embodiment
2.1 System configuration example
2.2 Configuration example of an image sensor
2.3 Configuration example of an EVS
2.4 Description of problems
2.5 Configuration example of a detection optical system
2.6 Specific example
2.7 Summary
2.8 Modifications
2.8.1 First modification
2.8.2 Second modification
2.8.3 Third modification
3. Hardware configuration
4. Modifications

### 1. Schematic Configuration Example of a Particle Analysis System

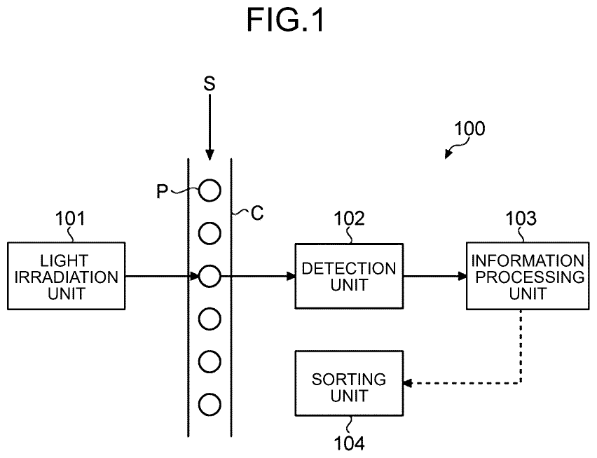

FIG. 1 illustrates a schematic configuration example of a particle analysis system according to a disclosure. A particle analysis system 100 illustrated in FIG. 1 is a biological sample analysis system and includes, for example, a light irradiation unit 101 that irradiates a biological sample S flowing through a channel C in a container with light, a detection unit 102 that detects light generated by the irradiation, and an information processing unit 103 that processes information concerning the light detected by the detection unit 102. Examples of the particle analysis system 100 include a flow cytometer and an imaging flow cytometer. The particle analysis system 100 includes a sorting unit 104 that sorts a specific microparticle (in the present explanation, referred to as bioparticle) P in the biological sample S. Examples of the particle analysis system 100 including the sorting unit 104 include a cell sorter. Note that the particle analysis system 100 may be replaced with a biological sample analysis system as appropriate and the channel C may be replaced with a container as appropriate in a range without contradiction.

### (Biological Sample)

The biological sample S may be a liquid sample containing bioparticles P. The bioparticles P are, for example, cells or non-cellular bioparticles. The bioparticles P may be microorganisms such as yeast or bacteria. The cells may be living cells and more specific examples the cells include blood cells such as red blood cells and white blood cells and germ cells such as sperms and fertilized eggs. The cells may be cells directly collected from a specimen such as whole blood or may be cultured cells acquired after culture. Examples of the non-cellular bioparticles include extracellular vesicles, in particular, exosomes and microvesicles. The bioparticles P may be labeled with one or a plurality of labeling substances (for example, a dye (in particular, a fluorescent dye) and a fluorescent dye labeled antibody). Note that, by the particle analysis system 100 of the present disclosure, particles other than the bioparticles may be analyzed or beads or the like may be analyzed for calibration or the like.

### (Channel)

The channel C can be configured such that the biological sample S flows, in particular, a flow in which the bioparticles P contained in the biological sample S are disposed substantially in a row is formed. A channel structure including the channel C may be designed such that a laminar flow is formed and, in particular, is designed such that a laminar flow in which the flow of the biological sample S (a sample flow) is wrapped by a flow of sheath liquid is formed. The design of the channel structure may be appropriately selected by those skilled in the art or a known channel structure may be adopted. The channel C may be formed in a channel structure such as a microchip (a chip including a channel in micrometer order) or a flow cell. The width of the channel C is 1 mm (millimeter) or less and may be, in particular, 10 μm (micrometer) or more and 1 mm or less. The channel C and the channel structure including the channel C may be formed of a material such as plastic or glass.

The device of the present disclosure may be configured such that the biological sample S flowing in the channel C, in particular, the bioparticles P in the biological sample S are irradiated with light from the light irradiation unit 101. The device of the present disclosure may be configured such that an irradiation point (Interrogation Point) of light with respect to the biological sample S is present in the channel structure in which the channel C is formed or may be configured such that the irradiation point of light is present outside the channel structure. Examples of the former can include a configuration in which the channel C in the microchip or the flow cell is irradiated with the light. In the latter, the bioparticles P after exiting the channel structure (in particular, a nozzle section thereof) may be irradiated with the light. Examples of the latter can include a flow cytometer of a jet in air type.

### (Light Irradiation Unit)

The light irradiation unit 101 includes a light source unit for detection that emits light and a light guide optical system that guides the light to the channel C. The light source unit for detection includes one or a plurality of light sources. A type of the light source can be, for example, a laser light source or an LED (Light Emitting Diode). A wavelength of light emitted from the light sources may be a wavelength of any one of ultraviolet light, visible light, or infrared light. The light guide optical system includes an optical component such as a beam splitter group, a mirror group, or an optical fiber. The light guide optical system may include a lens group for condensing light and can include, for example, an objective lens. The biological sample S may be irradiated with light at one or a plurality of irradiation points. The light irradiation unit 101 may be configured to condense light irradiated from one or a plurality of different light sources with respect to one irradiation point.

### (Detection Unit)

The detection unit 102 includes at least one photodetector that detects light generated by light irradiation on particles by the light irradiation unit 101. Light to be detected is, for example, fluorescence, scattered light (for example, any one or more of forward-scattered light, backscattered light, and side-scattered light), transmitted light, or reflected light. Each photodetector includes one or more light receiving elements and includes, for example, a light receiving element array. Each photodetector may include, as the light receiving elements, one or a plurality of photodiodes such as photomultiplier tubes (PMTs) and/or APDs (Avalanche Photodiodes) and MPPCs (Multi-Pixel Photon Counters). The photodetector includes, for example, a PMT array in which a plurality of PMTs are arrayed in a one-dimensional direction. The detection unit 102 may include an imaging element such as a CCD (Charge Coupled Device) or a CMOS (Complementary Metal-Oxide-Semiconductor). The detection unit 102 can acquire bioparticle information concerning the bioparticles P with the imaging element.

As explained above, the bioparticle information can include at least one of a bioparticle image of the bioparticles, a feature value of the bioparticles, attribute information of the bioparticles, and the like. The bioparticle image of the bioparticles may include, for example, a bright field image, a dark field image, a fluorescence image, and the like.

The detection unit 102 includes a detection optical system that causes light having a predetermined detection wavelength to reach a photodetector corresponding to the light. The detection optical system includes a spectroscopic unit such as a prism or a diffraction grating or a wavelength separation unit such as a dichroic mirror or an optical filter. The detection optical system may be configured, for example, to spectrally disperse light from the bioparticles P such that light in different wavelength regions is detected by a plurality of photodetectors larger in number than the number of fluorescent dyes. A flow cytometer including such a detection optical system is referred to as spectral flow cytometer. For example, the detection optical system may be configured to separate light corresponding to a fluorescence wavelength region of the fluorescent dyes from light from the bioparticles P and cause a photodetector corresponding to the separated light to detect the separated light.

The detection unit 102 can include a signal processing unit that converts an electric signal obtained by the photodetector into a digital signal. The signal processing unit may include an A/D converter as a device that performs the conversion. A digital signal obtained by the conversion by the signal processing unit can be transmitted to the information processing unit 103. The digital signal can be treated as data concerning light (hereinafter referred to as “optical data” as well) by the information processing unit 103. The optical data may be, for example, optical data including fluorescence data. More specifically, the optical data may be light intensity data and the light intensity may be light intensity data (which may include feature values such as Area, Height, and Width) of light including fluorescence.

### (Information Processing Unit)

The information processing unit 103 includes, for example, a processing unit that executes processing of various data (for example, optical data) and a storage unit that stores the various data. When acquiring the optical data corresponding to the fluorescent dyes from the detection unit 102, the processing unit can perform fluorescence leakage correction (compensation processing) on the light intensity data. In the case of the spectral flow cytometer, the processing unit executes fluorescence separation processing on the optical data and acquires light intensity data corresponding to the fluorescent dyes.

The fluorescence separation processing may be performed according to, for example, an un-mixing method described in Japanese Patent Application Laid-Open No. 2011-232259. When the detection unit 102 includes an imaging element, the processing unit may acquire form information of the bioparticles P based on an image acquired by the imaging element. The storage unit may be configured to be able to store the acquired optical data. The storage unit may be further configured to be able to store spectral reference data used in the un-mixing processing.

The particle analysis system 100 includes a sorting unit 104 explained below. The information processing unit 103 can determine, based on the optical data and/or the form information, whether to sort the bioparticles P. The information processing unit 103 controls the sorting unit 104 based on a result of the determination. The bioparticles P can be sorted by the sorting unit 104.

The information processing unit 103 may be configured to be able to output various data (for example, optical data and images). For example, the information processing unit 103 can output various data (for example, two-dimensional plots, spectral plots, and the like) generated based on the optical data. The information processing unit 103 may be configured to be able to receive input of various data and, for example, receives gating processing on a plot by a user. The information processing unit 103 can include an output unit (for example, a display) or an input unit (for example, a keyboard) for executing the output or the input.

The information processing unit 103 may be configured as a general-purpose computer and may be configured as an information processing device including, for example, a CPU (Central Processing Unit), a RAM (Random Access Memory), and a ROM (Read only memory). The information processing unit 103 may be included in a housing including the light irradiation unit 101 and the detection unit 102 or may be present outside the housing. Various kinds of processes or functions by the information processing unit 103 may be implemented by a server computer or a Cloud connected via a network.

### (Sorting Unit)

The sorting unit 104 can execute sorting of the bioparticles P, for example, according to a determination result by the information processing unit 103. A sorting scheme may be a scheme of generating droplets containing the bioparticles P with vibration, applying electric charges to the droplets to be sorted, and controlling a traveling direction of the droplets with an electrode. The sorting scheme may be a scheme of controlling the traveling direction of the bioparticles P in a channel structure to perform sorting. In the channel structure, for example, a control mechanism by pressure (injection or suction) or electric charges is provided. Examples of the channel structure include a chip (for example, a chip described in Japanese Patent Application Laid-Open No. 2020-76736) including a channel structure in which the channel C branches into a collection channel and a waste liquid channel on the downstream side thereof, specific bioparticles P being collected in the collection channel.

### 2. Embodiment

Subsequently, a particle analysis system, a particle analysis method, and a flow cytometer system according to an embodiment of the present disclosure are explained in detail with reference to the drawings.

### 2.1 System Configuration Example

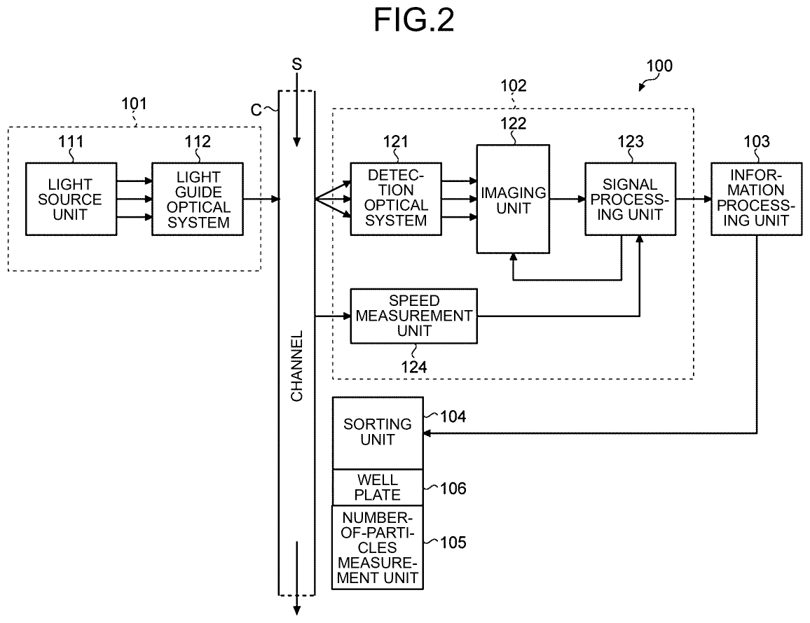

FIG. 2 is a block diagram illustrating a more specific configuration example of the particle analysis system according to the present embodiment. In the present embodiment and embodiments explained below, the particle analysis system may be configured as a system in which a plurality of devices are combined.

As illustrated in FIG. 2, the particle analysis system 100 according to the present embodiment includes a light source unit for detection 111 and a light guide optical system 112 configuring the light irradiation unit 101, a detection optical system 121, an imaging unit 122, a signal processing unit 123, and a speed measurement unit 124 configuring the detection unit 102, the information processing unit 103, the sorting unit 104, and a number-of-particles measurement unit 105. The particle analysis system 100 observes, in real time, images of fluorescence reflected light, and/or transmitted light emitted from the bioparticles P in the biological sample S flowing through the channel C, and sorts the target bioparticles P into wells of the well plate 106 based on a result of the observation. Then, the number of bioparticles P sorted into the wells of the well plate 106 is measured by the number-of-particles measurement unit 105. Note that the light source unit for detection 111, the light guide optical system 112, the detection optical system 121, the information processing unit 103, and the sorting unit 104 may be the same as those explained above with reference to FIG. 1.

More specifically, light (hereinafter also referred to as excitation light) output from the light source unit for detection 111 is condensed by the light guide optical system 112. The condensed light is applied to the bioparticles P flowing at high speed in the channel C through which the biological sample S in which the bioparticles P float are fed. Reflected light or transmitted light and/or fluorescence emitted from the bioparticles P irradiated with light is imaged on a light receiving surface of the imaging unit 122 through the detection optical system 121.

The imaging unit 122 includes, for example, pixels arrayed in a two-dimensional lattice pattern. For the imaging unit 122, various sensors such as a frame-type image sensor that outputs image data (also referred to as frame data) at a predetermined frame rate, an EVS (Event-based Vision Sensor) in which event pixels that detect an event based on a change in luminance of incident light are arrayed in a two-dimensional lattice pattern, and an image sensor of a so-called time delay integration (TDI) scheme that transfers a detection value of a line synchronized with the speed of the bioparticles P to an adjacent line and integrates the detection value may be used. The EVS used in the imaging unit 122 may be an EVS (see Japanese Patent Application No. 2020-191481) used in a configuration in which TDI processing is performed in a software manner using a timestamp included in event data. Alternatively, the image sensor of the time delay integration scheme used in the imaging unit 122 may be a TDI-PAD sensor (see Japanese Patent Application No. 2021-110227) configured by SPAD (Single-Photon Avalanche Diode) pixels that detect incidence of one photon. The matters described in Japanese Patent Application No. 2021-110227 and Japanese Patent Application No. 2020-191481 may be referred to as appropriate in the present disclosure.

Note that, as explained in detail below, the EVS may be a sensor that outputs event data including position information (an X address and a Y address) of a pixel in which an event has been detected, polarity information (a positive event/a negative event) of the detected event, information (a timestamp) of time when the event has been detected, and the like in a synchronous or asynchronous manner instead of the frame data.

Frame data acquired at a predetermined frame rate in the imaging unit 122 or a series of event data (hereinafter, also referred to as an event stream) generated in pixels to correspond to an image of the bioparticles P moving on the light receiving surface of the imaging unit 122 is transmitted to the signal processing unit 123.

The speed measurement unit 124 measures, for example, relative speed of the bioparticles P flowing through the channel C with respect to the speed measurement unit 124. In this example, since a case in which the speed measurement unit 124 is stationary with respect to the channel C is exemplified, in the following explanation, the speed measurement unit 124 is referred to as measuring the speed of the bioparticles P.

For the speed measurement unit 124, various detection schemes capable of detecting the speed of the bioparticles P such as an electrostatic scheme and an optical scheme may be adopted. The speed of the bioparticles P detected by the speed measurement unit 124 is sent to the signal processing unit 123 at any time.

Note that the speed measurement unit 124 may be omitted when the speed of the bioparticles P is known, for example, when the speed of the bioparticles P flowing through the channel C is controlled to be maintained at desired speed by controlling a pump system that delivers the biological sample S. However, even when the speed of the bioparticles P is known, the speed of the bioparticles P can fluctuate because of ambient temperature, a change in resistance of a liquid delivery system, or the like. Therefore, the speed of the bioparticles P may be actually measured using the speed measurement unit 124.

For example, when the imaging unit 122 is a frame-type image sensor, the signal processing unit 123 executes predetermined processing such as white balance adjustment and distortion correction on input frame data and sends the processed frame data to the information processing unit 103. On the other hand, when the imaging unit 122 is an EVS, the signal processing unit 123 reconstructs the frame data of the image of the bioparticles P from the event stream input from the imaging unit 122 and the speed of the bioparticles P, and sends the reconstructed frame data to the information processing unit 103. Further, when the imaging unit 122 is an image sensor of a time delay integration scheme, the signal processing unit 123 configures frame data from a predetermined number of lines and sends the configured frame data to the information processing unit 103.

Note that, when the imaging unit 122 is the EVS, a speed change of the bioparticles P is sufficiently gentle compared with an arrival frequency of the bioparticles P. Therefore, the speed of the bioparticles P used for reconfiguring the frame data is not limited to the speed of the bioparticles P themselves included in the frame data to be reconfigured and may be the velocity, an average value, or the like of the bioparticles P arriving before and/or after the bioparticles P.

The information processing unit 103 analyzes the frame data input from the signal processing unit 123 and executes correction for offsetting the rotation of the bioparticles P moving in the channel C, extraction of a feature value of the bioparticles P, discrimination of a type of the bioparticles P, and the like. The information processing unit 103 may include a display unit and may present bioparticle information used for the analysis, a feature value based on a result of the analysis, statistical data, a discrimination result of the type, and the like to the user. Further, the information processing unit 103 may sort and collect the bioparticles P of a specific type by controlling the sorting unit 104 based on the type discrimination result of the bioparticles P.

The sorting unit 104 sorts the bioparticles P moving in the channel C into the wells of the well plate 106 one by one based on the type discrimination result of the bioparticles P by the information processing unit 103. The bioparticles P of the specific type are sorted and collected in the wells of the well plate 106.

As explained in detail below, the number-of-particles measurement unit 105 includes a light source unit, an imaging unit, and a scanning mechanism and measures the number of bioparticles P sorted into the wells by scanning the wells of the well plate 106 in a depth direction (also referred to as a vertical direction) while illuminating the wells of the well plate 106 with the light source unit.

### 2.2 Configuration Example of an Image Sensor

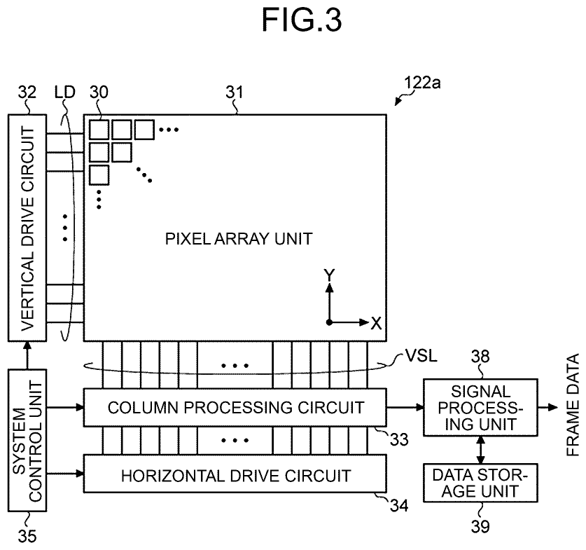

Here, a schematic configuration example of a frame-type image sensor that can be used as the imaging unit 122 is explained. FIG. 3 is a block diagram illustrating a schematic configuration example of a frame-type image sensor according to the present embodiment. Note that, in the present example, a CMOS (Complementary Metal-Oxide-Semiconductor) type image sensor is exemplified. However, the image sensor is not limited to this and may be various image sensors capable of acquiring color or monochrome image data such as a CCD (Charge-Coupled Device) type. The CMOS type image sensor may be an image sensor created by applying or partially using a CMOS process.

As illustrated in FIG. 3, an image sensor 122a has, for example, a stack structure in which a semiconductor chip on which the pixel array unit 31 is formed and a semiconductor chip on which a peripheral circuit is formed are stacked. The peripheral circuit may include, for example, a vertical drive circuit 32, a column processing circuit 33, a horizontal drive circuit 34, and a system control unit 35.

The image sensor 122a further includes a signal processing unit 38 and a data storage unit 39. The signal processing unit 38 and the data storage unit 39 may be provided on the semiconductor chip on which the peripheral circuit is provided or may be provided on another semiconductor chip.

The pixel array unit 31 has a configuration in which pixels 30 including photoelectric conversion elements that generate and accumulate electric charges corresponding to an amount of received light are disposed in a row direction and a column direction, that is, in a two-dimensional lattice shape in a matrix. Here, the row direction refers to an array direction of pixels in a pixel row (in the drawings, the lateral direction) and the column direction refers to an array direction of pixels in a pixel column (in the drawings, the longitudinal direction).

In the pixel array unit 31, pixel drive lines LD are wired along the row direction for each of pixel rows and vertical signal lines VSL are wired along the column direction for each of pixel columns with respect to a matrix-like pixel array. The pixel drive lines LD transmit a drive signal for performing driving in reading a signal from the pixels. In FIG. 3, each the pixel drive lines LD is illustrated as one wiring line but is not limited to the one wiring line. One ends of the pixel drive lines LD are connected to output terminals corresponding to the rows of the vertical drive circuit 32.

The vertical drive circuit 32 includes a shift register, an address decoder, and the like and drives the pixels of the pixel array unit 31, for example, simultaneously for all pixels or in units of rows. That is, the vertical drive circuit 32 configures, in conjunction with the system control unit 35 that controls the vertical drive circuit 32, a drive unit that controls the operation of the pixels of the pixel array unit 31. Although a specific configuration of the vertical drive circuit 32 is not illustrated, the vertical drive circuit 32 generally includes two scanning systems, that is, a read scanning system and a sweep scanning system.

In order to read a signal from the pixels 30, the read scanning system sequentially selectively scans the pixels 30 of the pixel array unit 31 in units of rows. The signal read from the pixels 30 is an analog signal. The sweep scanning system performs sweep scanning on a read row, on which read scanning is performed by the read scanning system, prior to the read scanning by an exposure time.

By the sweep scanning by the sweep scanning system, unnecessary electric charges are swept out from the photoelectric conversion elements of the pixels 30 in the read row, whereby the photoelectric conversion elements are reset. Then, by sweeping out (resetting) the unnecessary electric charges in the sweep scanning system, a so-called electronic shutter operation is performed. Here, the electronic shutter operation refers to an operation of discarding electric charges of the photoelectric conversion element and starting exposure (starting accumulation of electric charges) anew.

A signal read by the read operation by the read scanning system corresponds to an amount of light received after the immediately preceding read operation or the electronic shutter operation. Then, a period from the read timing by the immediately preceding read operation or the sweep timing by the electronic shutter operation to the read timing by the current read operation is a charge accumulation period (also referred to as exposure period) in the pixels 30.

A signal output from the pixels 30 of the pixel row selectively scanned by the vertical drive circuit 32 is input to the column processing circuit 33 through each of the vertical signal lines VSL for each of the pixel columns. The column processing circuit 33 performs predetermined signal processing on the signal output from the pixels of the selected row through the vertical signal line VSL for each of the pixel columns of the pixel array unit 31 and temporarily retains the pixel signal after the signal processing.

Specifically, the column processing circuit 33 performs at least noise removal processing, for example, CDS (Correlated Double Sampling processing or DDS (Double Data Sampling) as the signal processing. For example, fixed pattern noise specific to the pixels such as reset noise and threshold variation of amplification transistors in the pixels is removed by the CDS processing. Besides, the column processing circuit 33 also has, for example, an AD (analog-digital) conversion function and converts an analog pixel signal read from the photoelectric conversion element into a digital signal and outputs the digital signal.

The horizontal drive circuit 34 is configured by a shift register, an address decoder, and the like and selects, in order, read circuits (hereinafter referred to as pixel circuits) corresponding to the pixel columns of the column processing circuit 33. By the selective scanning by the horizontal drive circuit 34, pixel signals subjected to signal processing for each of the pixel circuits in the column processing circuit 33 are output in order.

The system control unit 35 is configured by, for example, a timing generator that generates various timing signals and performs drive control for the vertical drive circuit 32, the column processing circuit 33, the horizontal drive circuit 34, and the like based on various timings generated by the timing generator.

The signal processing unit 38 has at least an arithmetic processing function and performs various kinds of signal processing such as arithmetic processing on a pixel signal output from the column processing circuit 33. In the signal processing in the signal processing unit 38, the data storage unit 39 temporarily stores data necessary for the processing. Note that, when the signal processing unit 38 has calculation functions such as white balance adjustment and distortion correction, the signal processing unit 123 illustrated in FIG. 2 may be omitted.

### 2.3 Configuration Example of an EVS

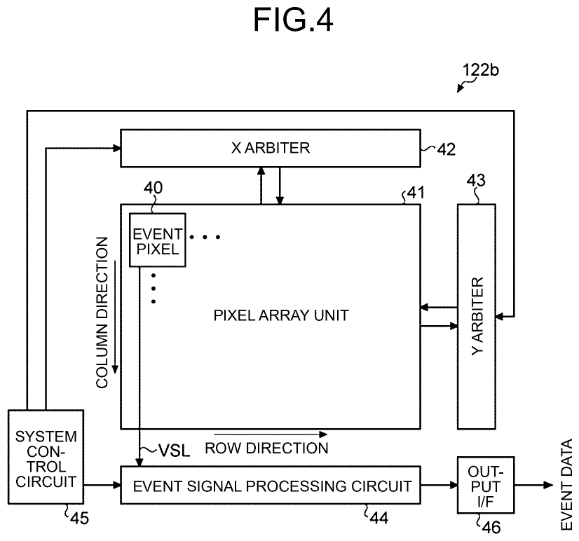

Subsequently, a schematic configuration example of the EVS that can be used as the imaging unit 122 is explained. FIG. 4 is a block diagram illustrating a schematic configuration example of an EVS according to the present embodiment. As illustrated in FIG. 4, an EVS 122b includes a pixel array unit 41, an X arbiter 42 and a Y arbiter 43, an event signal processing circuit 44, a system control circuit 45, and an output interface (I/F) 46.

The pixel array unit 41 has a configuration in which a plurality of event pixels 40, each of which detects an event based on a luminance change of incident light, are arrayed in a two-dimensional lattice pattern. Note that, in the following explanation, a row direction refers to an array direction of pixels of a pixel row (in the drawings, the lateral direction) and a column direction refers to an array direction of pixels in a pixel column (in the drawings, the longitudinal direction).

The event pixels 40 include photoelectric conversion elements that generate electric charges corresponding to the luminance of incident light. When a luminance change of the incident light is detected based on a photocurrent flowing out from the photoelectric conversion elements, the event pixels 40 output requests for requesting reading from the event pixel 40 to the X arbiter 42 and the Y arbiter 43 and output, according to arbitration by the X arbiter 42 and the Y arbiter 43, an event signal indicating that an event has been detected.

The event pixels 40 detect the presence or absence of an event based on whether a change exceeding a predetermined threshold has occurred in a photocurrent corresponding to the luminance of incident light. For example, the event pixels 40 detect, as an event, the luminance change exceeding the predetermined threshold (a positive event) or falling below the predetermined threshold (a negative event).

When detecting the event, the event pixels 40 output requests for requesting permission to output an event signal representing the occurrence of the event to each of the X arbiter 42 and the Y arbiter 43. Then, the event pixels 40 output event signals to the event signal processing circuit 44 when receiving a response representing permission to output the event signal from each of the X arbiter 42 and the Y arbiter 43.

The X arbiter 42 and the Y arbiter 43 arbitrate the request for requesting the output of the event signal supplied from each of the plurality of event pixels 40 and transmit a response based on a result of the arbitration (permission/non-permission of the output of the event signal) and a reset signal for resetting the event detection to the event pixel 40 that has output the request.

The event signal processing circuit 44 generates event data by executing predetermined signal processing on the event signal input from the event pixel 40 and outputs the event data.

As explained above, the change in the photocurrent generated in the event pixel 40 can also be grasped as a light amount change (a luminance change) of light made incident on the photoelectric conversion unit of the event pixel 40. Therefore, it can also be considered that the event is a light amount change (a luminance change) of the event pixel 40 exceeding a predetermined threshold. The event data representing the occurrence of the event includes at least position information such as coordinates indicating the position of the event pixel 40 where the light amount change as the event has occurred. The event data can include polarity of a light amount change besides the position information.

For a series of event data output from the event pixel 40 at the timing when the event has occurred, as long as the interval among the event data is maintained at an interval at the time of the occurrence of the event, it can be considered that the event data implicitly includes time information representing relative time when the event has occurred.

However, when the interval among the event data is not maintained as the interval at the time of the occurrence of the event because, for example, the event data is stored in a memory or the like, the time information implicitly included in the event data is lost. Therefore, before the interval among the event data is not maintained as the interval at the time of the occurrence of the event, the event signal processing circuit 44 may include time information indicating relative time when the event has occurred such as a timestamp in the event data.

### (Other Components)

The system control circuit 45 is configured by, for example, a timing generator that generates various timing signals and performs drive control for the X arbiter 42, the Y arbiter 43, the event signal processing circuit 44, and the like based on various timings generated by the timing generator.

The output I/F 46 sequentially outputs event data output in units of rows from the event signal processing circuit 44 to the signal processing unit 123 as an event stream. In response to the output, the signal processing unit 123 generates image data (also referred to as frame data) having a predetermined frame rate by accumulating the event data input as the event stream for a predetermined frame period.

### 2.4 Description of Problems

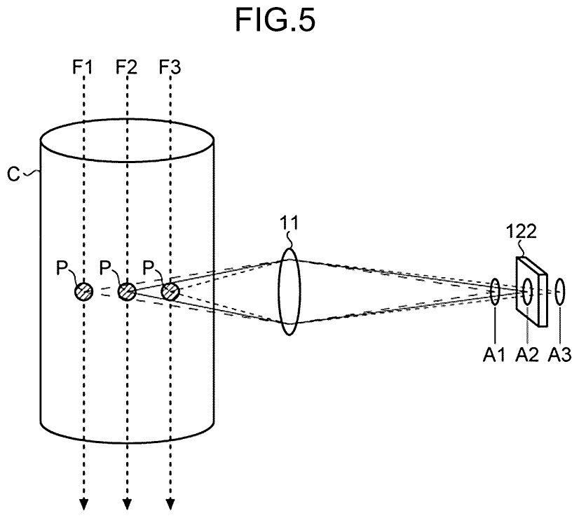

In the particle analysis system explained above that acquires an image of the bioparticles P flowing in the channel C, as illustrated in FIG. 5, a passing positions of the bioparticles P in the channel C are random. For that reason, it is likely that the passing position of the bioparticles P in the channel C deviates from a focal position of an objective lens 11 in the detection optical system 121 and an out-of-focus unclear image is acquired. For example, when the focal position of the objective lens 11 is adjusted to a path F2 passing the center of the channel C, an image obtained by imaging the bioparticles P deviating from the path F2, for example, passing a path F1 or a path F2 becomes an unclear image that is out of focus with respect to the bioparticles P.

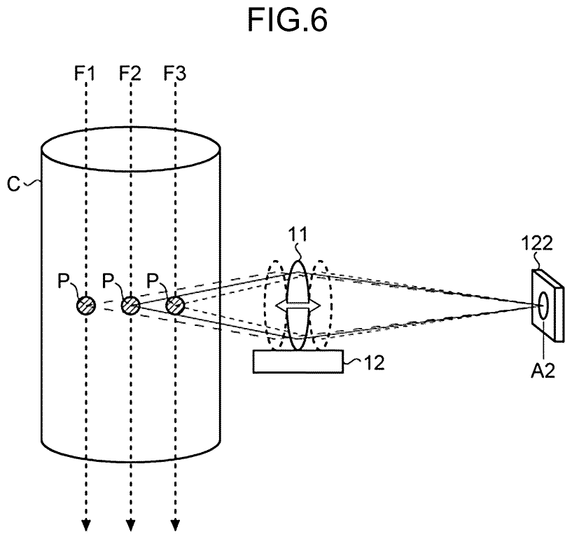

As a method of coping with such a problem, as illustrated in FIG. 6, it is conceivable to measure a passing position of the bioparticles P in the channel, and perform focus adjustment for moving the objective lens 11 using a moving mechanism 12 to adjust the focal position of the objective lens 11 on a passing path of the bioparticles P such that the individual passing positions are focused.

However, in a method of physically moving an optical element such as the objective lens 11 for focus adjustment as explained above, there is a problem in that adjustment speed is extremely low with respect to the speed of the bioparticles P. Therefore, it is necessary to align the focal position with the passing position of the bioparticles P by the time of imaging by measuring in advance an indicator highly correlated with the deviation between the focal position and the passing position of the bioparticles P prior to the imaging of the bioparticles P and starting driving of a focus mechanism in advance based on a result of the measurement.

However, in such a method, there are problems that an error occurs due to the fact that the correlation between the measured indicator and the deviation of the passing position of the bioparticles P from the actual focal position does not completely match and that, since it takes time to change the focal position of the detection optical system 121, when the number of bioparticles P increases and an imaging interval is shortened, focus adjustment for the individual bioparticles P cannot be performed in time.

For example, Patent Literature 1 discloses a method of detecting a deviation from a focal position of a cell from a combination of time waveforms of the intensities of scattered light from the cell transmitted through a pair of gratings. Patent Literature 1 proposes a method of estimating the subsequent transition from a tendency of a temporal change of detected focal deviation and moving a lens such that a focal position comes to an estimated value. However, in this method, since an estimation amount depends on the size and shape of the cell, there is a problem in that it is difficult to generically maintain sufficient accuracy.

For that reason, the present embodiment makes it possible to acquire a plurality of images having different focal positions in a process in which the bioparticles P flow. An image in focus is selected out of the images. As a result, it is unnecessary to perform focus adjustment on the individual bioparticles P. Therefore, even when the number of the bioparticles P increases and an imaging interval is shortened, it is possible to suppress deterioration in image quality due to deviation of the passing position of the bioparticles P from the focal position. Since focus adjustment based on an indicator having a high correlation with the deviation between the focal position and the passing position of the bioparticles P is unnecessary, it is also possible to suppress occurrence of an error due to a matching degree of a correlation.

### 2.5 Configuration Example of a Detection Optical System

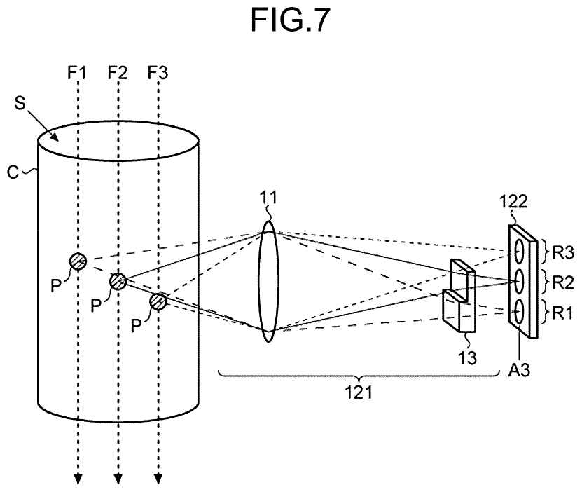

FIG. 7 is a diagram illustrating a schematic configuration example of a detection optical system according to the present embodiment. As illustrated in FIG. 7, the detection optical system 121 according to the present embodiment includes, for example, the objective lens 11 and an optical path length adjustment element 13. Optical elements included in the detection optical system 121 are not limited these elements and the detection optical system 121 may include various optical elements such as a collimator lens, a diaphragm, and a spectroscopic element.

The focal position of the objective lens 11 is adjusted to a predetermined position in the channel C. For example, the focal position of the objective lens 11 is adjusted on the path F2 passing through the center in the channel C.

The optical path length adjustment element 13 is disposed, for example, on an optical path between the objective lens 11 and the imaging unit 122. Note that, when the detection optical system 121 includes an optical element other than the objective lens 11, the optical path length adjustment element 13 may be disposed at any position on the optical path between the objective lens 11 and the imaging unit 122.

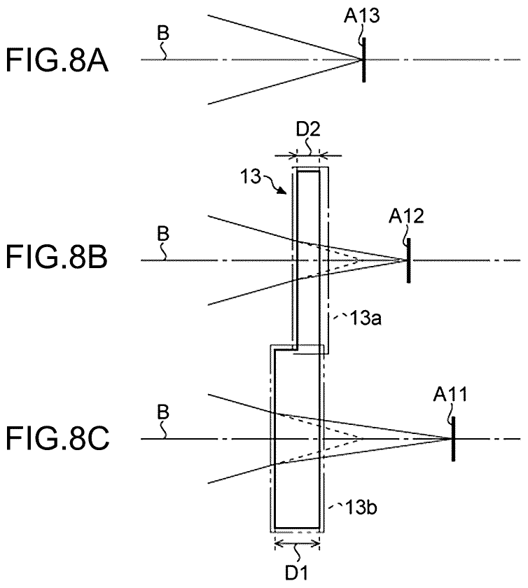

FIG. 8 is a diagram for explaining the principle of the optical path length adjustment element according to the present embodiment. As illustrated in FIG. 8, the optical path length adjustment element 13 is made of, for example, a glass plate having different thicknesses stepwise. However, as the material of the optical path length adjustment element 13, various materials may be adopted if the materials transmit observation light (fluorescence, scattered light, or the like) from the bioparticles P and have a refractive index different from the refractive index in the air or vacuum.

As explained above, by using the optical path length adjustment element 13 having the refractive index different from the refractive index in the air or vacuum and having thicknesses different stepwise for each of the regions, as illustrated in FIG. 8, an image forming position of an image of observation light transmitted through regions can be changed according to the thickness of the optical path length adjustment element 13. This makes use of a principle that a condensing position of light transmitted through a medium having a refractive index different from the refractive index of the surrounding environment (the air or vacuum) changes from a condensing position of light not passing through the medium.

As in this example, in a case where a material having a refractive index higher than the ambient environment (air or vacuum) such as a glass plate is used for the optical path length adjustment element 13, as illustrated in FIG. 8, image forming positions A11 and A12 of the light transmitted through the optical path length adjustment element 13 become farther as the thickness of the optical path length adjustment element 13 is thicker ((B)<(C) in FIG. 8) than an image forming position A13 (see (A) of FIG. 8) of the observation light that does not transmit through the optical path length adjustment element 13.

On the other hand, as illustrated in FIG. 5, when the focal position of the objective lens 11 is set on the path F2 in the channel C and the light receiving surface of the imaging unit 122 is disposed at the image forming position A2 via the objective lens 11 of the image of the bioparticles P passing through the path F2, an image forming position A1 of an image of the bioparticles P passing through the path F1, which is more distant from the objective lens 11 than the path F2, is further on the front side (the objective lens 11 side) than an image forming position A2 of an image of the bioparticles P passing through the path F2 and an image forming position A3 of an image of the bioparticles P passing through a path F3, which is closer from the objective lens 11 than the path F2 is further on the rear side of the image forming position A2 of the image of the bioparticles P passing through the path F2 (the side more distant from the objective lens 11 than the light receiving surface of the imaging unit 122). That is, the image of the bioparticles P passing through the position more distant than the path F2 passing the focal position of the objective lens 11 is formed further on the front side than the light receiving surface of the imaging unit 122 and the image of the bioparticles P passing the position closer than the path F2 is formed further on the rear side than the light receiving surface of the imaging unit 122.

Therefore, in the present embodiment, as illustrated in FIG. 7, the optical path length adjustment element 13 having the refractive index different from the refractive index of the air or the like and having the thickness different stepwise for each of the regions is disposed on the optical path between the objective lens 11 and the imaging unit 122 to have a positional relation in which the thickness changes stepwise along the flowing direction of the bioparticles P. Accordingly, in a process in which the bioparticles P flowing in the channel C pass through the objective lens 11 and an image of the bioparticles P is formed on the light receiving surface of the imaging unit 122, an imaging distance of the image of the bioparticles P can be changed in several stages from the upper side (corresponding to the downstream side in the channel C) to the lower side (corresponding to the upstream side in the channel C) within the angle of view of the imaging unit 122.

At that time, when the refractive index of the optical path length adjustment element 13 is higher than the refractive index of a surrounding environment (air, vacuum, or the like), the light receiving surface of the imaging unit 122 may be disposed at the image forming position A3 of an image not transmitted through the optical path length adjustment element 13 (that is, an image of the bioparticles P passing through the path F3 at the shortest distance from the objective lens 11). However, when the refractive index of the optical path length adjustment element 13 is lower than the refractive index of the surrounding environment (air, vacuum, or the like), the light receiving surface of the imaging unit 122 only has to be disposed at the image forming position Al of the image transmitted through the thickest region in the optical path length adjustment element 13 (that is, the image of the bioparticles P passing through the path F1 most distance from the objective lens 11).

In this explanation, for simplicity, it is assumed that the refractive index of the optical path length adjustment element 13 is higher than the refractive index of the surrounding environment (air, vacuum, or the like) and the thickness of the optical path length adjustment element 13 changes in two stages of a thin region (also referred to as first portion) 13a and a thick region (also referred to as second portion) 13b.

In such a configuration, for example, when a region having a larger thickness in the optical path length adjustment element 13 is disposed on the lower side (equivalent to the upstream side with respect to the channel C) within the angle of view of the imaging unit 122 via the objective lens 11 and the optical path length adjustment element 13 is not disposed on the upper side (equivalent to the downstream side with respect to the channel C) within the angle of view of the imaging unit 122 via the objective lens 11, an image of the bioparticles P located on the upstream side of the channel C in the visual field of the objective lens 11 is transmitted through the second portion 13b of the optical path length adjustment element 13 and formed in a region R3 on the downstream side of the imaging unit 122. An image of the bioparticles P located near the center in the visual field of the objective lens 11 is transmitted through the first portion 13a of the optical path length adjustment element 13 and formed in a region R2 near the center of the imaging unit 122. An image of the bioparticles P located on the downstream side of the channel C in the visual field of the objective lens 11 is formed in the region R3 on the upstream side of the imaging unit 122 without being transmitted through the optical path length adjustment element 13.

The imaging unit 122 sequentially generates image data of images formed in order in a region R1, the region R2, and the region R3 and outputs the generated image data to the signal processing unit 123.

As explained above, for example, when the imaging unit 122 is the frame-type image sensor, the signal processing unit 123 executes the predetermined processing such as the white balance adjustment and the distortion correction on the input frame data and sends the processed frame data to the information processing unit 103. On the other hand, when the imaging unit 122 is the EVS, the signal processing unit 123 reconfigures the frame data of the image of the bioparticles P from the event stream input from the imaging unit 122 and the speed of the bioparticles P and sends the reconfigured frame data to the information processing unit 103. Further, when the imaging unit 122 is the image sensor of the time delay integration scheme, the signal processing unit 123 configures the frame data from the predetermined number of lines and sends the configured frame data to the information processing unit 103.

The information processing unit 103 evaluates a focus state of the images with respect to the bioparticles P from the sequentially input image data (an evaluation unit). Here, the passing position of the bioparticles P in the channel C and the sharpness of the image of the bioparticles P in the image data are explained.

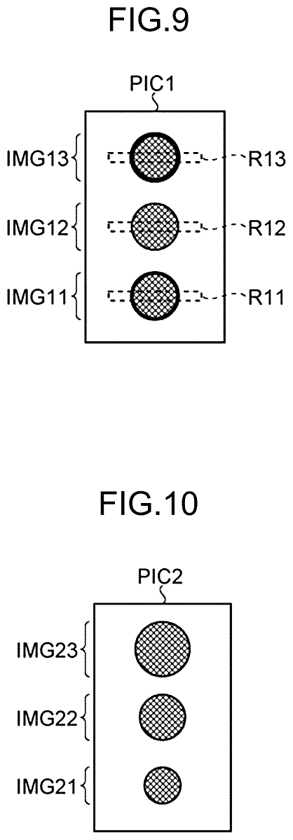

FIG. 9 and FIG. 10 are diagrams for explaining a method of evaluating a focus state by the information processing unit according to the present embodiment. Note that, in FIG. 9 and FIG. 10, for clarity, images of the bioparticles P present at different positions on a path are displayed to be superimposed on one piece of image data. However, the image data of the images may be separate image data (frame data).

FIG. 9 illustrates an example of image data acquired when the bioparticles P pass through the path F2 in FIG. 7. As illustrated in FIG. 9, when the bioparticles P pass through the path F2 passing the center of the channel C, an image of the bioparticles P located near the center in the visual field of the objective lens 11 is transmitted through the first portion 13a of the optical path length adjustment element 13 and formed in the region R2 near the center of the imaging unit 122. Therefore, an image IMG12 of the bioparticles P in image data PIC1 is a focused clear image.

On the other hand, an image of the bioparticles P located on the upstream side of the channel C in the visual field of the objective lens 11 passes through the second portion 13b located on the downstream side of the optical path length adjustment element 13 and is formed in the region R1 on the downstream side of the imaging unit 122. Therefore, an image IMG11 of the bioparticles P in the image data PIC1 is an unfocused unclear image. Similarly, an image of the bioparticles P located on the downstream side of the channel C in the visual field of the objective lens 11 passes through a region where the optical path length adjustment element 13 is absent located on the upstream side and is formed in the region R3 on the upstream side of the imaging unit 122. Therefore, an image IMG13 of the bioparticles P in the image data PIC1 is an unfocused unclear image. Therefore, when the bioparticles P pass through the path F2 passing the center of the channel C, an image IMG12 at the time when the bioparticles P are located near the center in the visual field of the objective lens 11 is a clearest image.

As illustrated in FIG. 10, when the bioparticles P pass through the path F1 more distant from the objective lens 11 in the channel C, an image IMG21 of the bioparticles P passing through the second portion 13b located on the downstream side of the optical path length adjustment element 13 and formed in the region R1 on the downstream side of the imaging unit 122 is a clearest image. When the bioparticles P pass through the path F2 passing the center of the channel C, an image IMG22 of the bioparticles P passing through the first portion 13a located on the upstream side of the optical path length adjustment element 13 and formed in the region R2 near the center of the imaging unit 122 is a clearest image. When the bioparticles P pass through the path F1 more distant from the objective lens 11 in the channel C, the image IMG21 of the bioparticles P passing through the second portion 13b located on the downstream side of the optical path length adjustment element 13 and formed in the region R1 on the downstream side of the imaging unit 122 is a clearest image.

Therefore, by analyzing the image data sequentially input from the imaging unit 122 via the signal processing unit 123, it is possible to evaluate a focus state of the images included in the image data with respect to the bioparticles P. Note that, as a method of evaluating the focus state, for example, various schemes generally used in an autofocus function of a camera or the like such as a contrast detection scheme or a phase difference detection scheme may be used. Specifically, for example, evaluation values described below may be calculated and an image having the highest result obtained by combining any one or two or more of these evaluation values may be evaluated as an image in the best focus state.
1. An integrated value of luminance differences among adjacent pixels in an entire or a part of region of image data (for example, a region equivalent to an image of the bioparticles P)
2. An intensity value of a high-frequency component in the entire or a part of region of the image data (for example, a region equivalent to the image of the bioparticles P)
3. An outer diameter dimension of the image of the bioparticles P in the image data

However, when the image of the bioparticles P passes through the second portion 13b located on the downstream side of the optical path length adjustment element 13, the optical path length is increased by the optical path length adjustment element 13. Therefore, the size of the image IMG21 in image data PIC2 is smaller than the size of the other images. When the image of the bioparticles P passes through a region where the optical path length adjustment element 13 is absent, the optical path length is not increased. Therefore, the size of an image IMG23 in the image data PIC2 is larger than the size the other images.

For that reason, in order to evaluate which image among images having different optical path lengths is the clearest, that is, to evaluate a focus state of which image is most satisfactory, it is necessary to evaluate a focus state targeting a portion corresponding to the same region of the bioparticles P in the same image of the bioparticles P. Therefore, in the present embodiment, as illustrated in FIG. 9, evaluation target regions R11 to R13 in image data may be specified based on a relation between speed at the time when the bioparticles P pass through the visual field of the objective lens 11 and an acquisition time of the image data.

With the above configuration, it is possible to acquire a series of image data including image data focused on the bioparticles P in a process in which the bioparticles P pass through any of the paths F1 to F3 from the upstream to the downstream. Then, by selecting image data including an image in a satisfactory focus state out of the series of image data, it is possible to select image data in which the objective lens 11 is focused on the bioparticles P. Therefore, it is possible to suppress deterioration in image quality due to deviation from a focal position.

Note that the image in the satisfactory focus state (which may be entire individual image data) selected as explained above may be used for processing such as analysis and identification of the bioparticles P in the information processing unit 103.

### 2.6 Specific Example

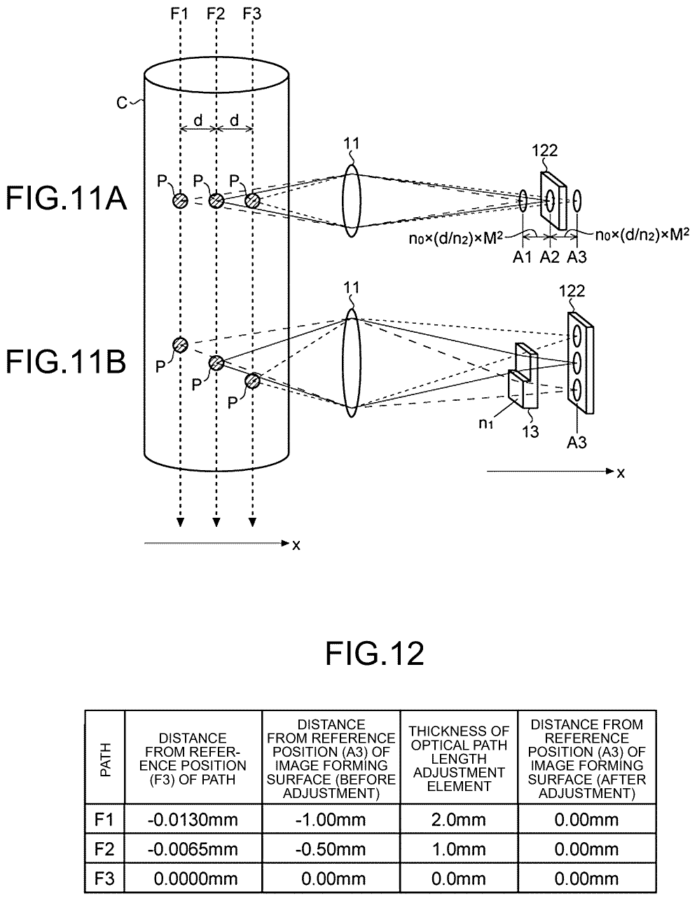

Subsequently, a specific example of an image of the bioparticles P acquired by the imaging unit 122 according to the present embodiment is explained. FIG. 11 and FIG. 12 are diagrams for explaining a specific example of an image of bioparticles acquired by the imaging unit according to the present embodiment.

As illustrated in FIG. 11, in this specific example, a case is exemplified in which an interval among the paths F1 to F3 in the channel C, that is, a difference (also referred to as distance or interval) among the distances of the paths F1 to F3 from the objective lens 11 is represented as d, a refractive index of liquid (for example, water) of the biological sample S flowing in the channel is represented as n₂, a refractive index of a surrounding environment (for example, air) is represented as n₀, a refractive index of the optical path length adjustment element 13 is represented as n₁, lateral magnification (that is, magnification in a direction perpendicular to an optical path) of the objective lens 11 is represented as M, and the image forming position A3 of the image of the bioparticles P passing through the path F3 is set as a reference position of an imaging plane.

In such a case, as illustrated in (A) of FIG. 11, an imaging distance via the objective lens 11 of the image of the bioparticles P passing through the path F2 more distant from the objective lens 11 by the distance d than the path F3 is shorter by n₀×(d/n₂)×M² obtained by multiplying n₀×(d/n₂) by vertical magnification (that is, magnification in the direction along the optical path) M² than an imaging distance via the objective lens 11 of the image of the bioparticles P passing through the path F3.

Therefore, in the present embodiment, as illustrated in (B) of FIG. 11, the optical path length adjustment element 13 (equivalent to the first portion 13a) is disposed on the optical path of the image of the bioparticles P in order to align the image forming position A2 of the image of the bioparticles P passing through the path F2 with the reference position (A3). At that time, by setting the thickness of the optical path length adjustment element 13 (the first portion 13a) to (n₀×(d/n₂)×M²)/(n₁−1), the imaging distance of the image of the bioparticles P passing through the path F2 is longer by n₀×(d/n₂)×M². Therefore, the image forming position A2 of the image of the bioparticles P passing through the path F2 can be aligned with the reference position (A3).

For example, as illustrated in FIG. 12, when it is assumed that the distance d is 6.5 μm, n₂ is 1.3, n₀ is 1.0, M is ten times, and n1 is 1.5, by setting the thickness of the optical path length adjustment element 13 (the first portion 13a) to (1.0×(0.0065/1.3)×10×10)/(1.5−1)=1.0 mm, the image forming position A2 of the image of the bioparticles P passing through the path F2 can be aligned with the reference position (A3).

Similarly, in order to align the image forming position A1 of the image of the bioparticles P passing through the path F1 with the reference position (A3), by disposing the optical path length adjustment element 13 (equivalent to the thick region) having thickness calculated with the distance set to 2d on the optical path of the image of the bioparticles P, the image forming position A1 of the image of the bioparticles P passing through the path F1 can be aligned with the reference position (A3).

For example, as illustrated in FIG. 12, by setting the thickness of the optical path length adjustment element 13 (the second portion 13b) to (1.0×(2×0.0065/1.3)×10×10)/(1.5−1)=2.0 mm, the image forming position A1 of the image of the bioparticles P passing through the path F1 can be aligned with the reference position (A3).

### 2.7 Summary

As explained above, in the present embodiment, the inside of the visual field of the objective lens 11 (the inside of the angle of view of the imaging unit 122) is divided into a plurality of regions. The optical path length of the light made incident on the respective regions (the images of the bioparticles P) is adjusted using the optical path length adjustment element 13. This makes it possible to match image forming positions of images passing through the regions with the light receiving surface of the imaging unit 122. Therefore, it is possible to acquire image data focused on each of the bioparticles P passing through the different paths in the channel C.

Note that, in the above explanation, a case is exemplified in which the visual field of the objective lens 11 (within the angle of view of the imaging unit 122) is divided into three regions using the optical path length adjustment element 13, the thickness of which changes in two stages. However, not only this, but the thickness of the optical path length adjustment element 13 may change in one stage (that is, the optical path length adjustment element 13 may be a flat plate) or in three or more stages (n stages). When the thickness of the optical path length adjustment element 13 changes in one stage, the inside of the visual field of the objective lens 11 (the inside of the angle of view of the imaging unit 122) may be divided into two regions. When the thickness of the optical path length adjustment element 13 changes in n stages (n is an integer of 3 or more), the inside of the visual field of the objective lens 11 (the inside of the angle of view of the imaging unit 122) may be divided into (n+1) regions.

The inside of the visual field of the objective lens 11 (the inside of the angle of view of the imaging unit 122) may be divided into more regions and the interval d among the focused paths may be set narrower (for example, smaller than the size of the bioparticles P.). This makes it possible to acquire a plurality of pieces of image data focused on one bioparticle P. In that case, since it is possible to combine images of different focus positions for each the regions of the bioparticles P to synthesize one or a plurality of pieces of image data, it is possible to generate combined images individually focused on a plurality of inclusions distributed at different positions in the focal direction in the bioparticles P. Alternatively, it is also possible to generate three-dimensional volume data of the bioparticles P from a plurality of pieces of image data having different focus positions.

When the interval d among the focused paths is set narrower than the size of the bioparticles P, the distance (equivalent to the distance d) between a focal position in the channel C of the detection optical system 121 for light transmitted through the detection optical system 121 to form an image on the light receiving surface of the imaging unit 122 without being transmitted through the optical path length adjustment element 13 and a focal position in the channel C of the detection optical system 121 for light transmitted through the detection optical system 121 and the optical path length adjustment element 13 to form an image on the light receiving surface of the imaging unit 122 is adjusted to be shorter than the diameter of the bioparticles P. Specifically, when the size of the bioparticles P is represented as L and a stepwise difference in the thickness of the optical path length adjustment element 13 is represented as D, D is desirably set to satisfy the following Expression (1).

$$D < ( {n}_{0} \times ( L / {n}_{2} ) \times {M}^{2} ) / ( {n}_{1} - 1 ) \qquad ( 1 )$$

### 2.8 Modifications

Subsequently, modifications of the present embodiment are explained with some examples.

### 2.8.1 First Modification

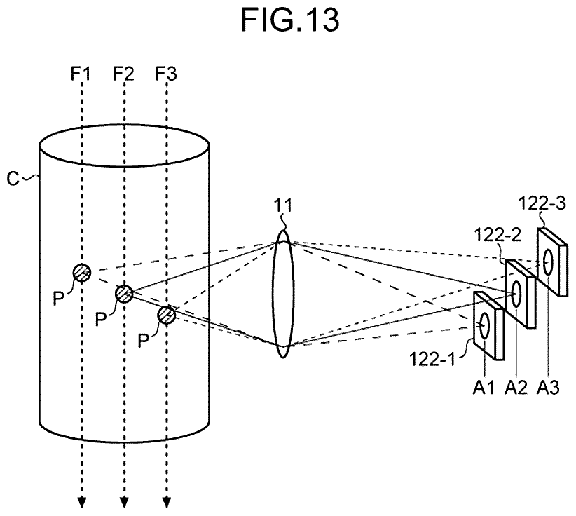

FIG. 13 is a diagram illustrating a schematic configuration example of a detection optical system according to a first modification of the present embodiment. In the above explanation, by disposing the optical path length adjustment element 13 on the optical path of the light (scattered light, fluorescence, or the like) from the bioparticles P, the image forming positions of the images of the bioparticles P passing the different positions in the channel C are matched. However, the present embodiment is not limited to this. For example, as illustrated in FIG. 13, by disposing imaging units 122-1 to 122-3 respectively at the image forming positions Al to A3 before adjustment by the optical path length adjustment element 13, image data focused on the bioparticles P passing through the different paths in the channel C may be acquired. In that case, each of the imaging units 122-1 to 122-3 may have the same configuration as the configuration of the imaging unit 122.

### 2.8.2 Second Modification

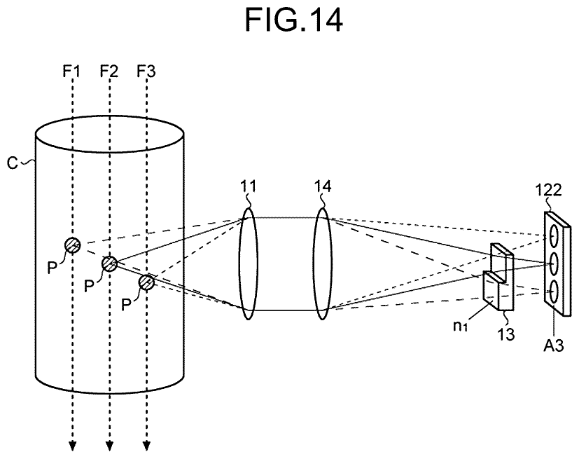

FIG. 14 is a diagram illustrating a schematic configuration example of a detection optical system according to a second modification of the present embodiment. In the above explanation, a case is exemplified in which the objective lens 11 forms the image of the bioparticles P on the light receiving surface of the imaging unit 122 directly or via the optical path length adjustment element 13. However, not only this, but, for example, as illustrated in FIG. 14, the image of the bioparticles P may be formed on the light receiving surface of the imaging unit 122 via an image forming lens 14 different from the objective lens 11. In that case, the optical path length adjustment element 13 may be disposed on an optical path between the image forming lens 14 and the imaging unit 122.

### 2.8.3 Third Modification

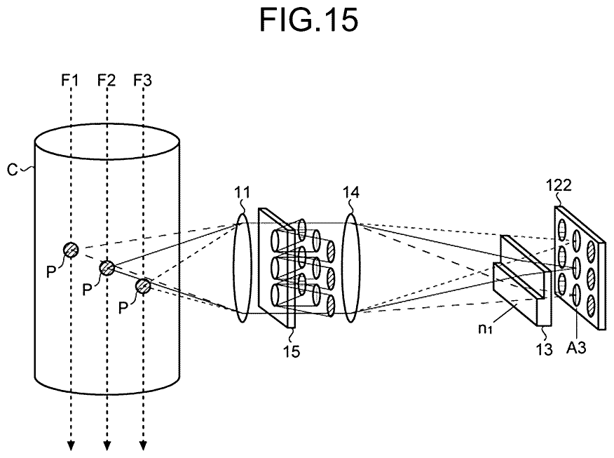

FIG. 15 is a diagram illustrating a schematic configuration example of a detection optical system according to a third modification of the present embodiment. As illustrated in FIG. 15, when the image of the bioparticles P is formed on the light receiving surface of the imaging unit 122 via the image forming lens 14 different from the objective lens 11, a spectroscopic element 15 may be disposed between the objective lens 11 and the image forming lens 14. The spectroscopic element 15 may be disposed to disperse light from the bioparticles P in a direction perpendicular to the moving direction of the bioparticles P. By dispersing the light (fluorescence in this example) from the bioparticles P and forming images for each of wavelengths at different positions of the imaging unit 122, it is also possible to acquire a fluorescence image for each of fluorescence wavelengths from the bioparticles P together with a bright field image or a dark field image of the bioparticles P including a wavelength of a detection light source.

### 3. Hardware Configuration

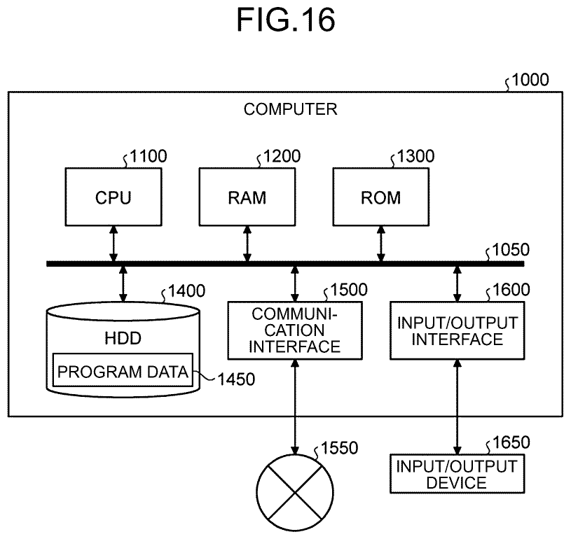

The signal processing unit 123 and the information processing unit 103 according to the embodiment and the modifications thereof explained above can be implemented by a computer 1000 having, for example, a configuration illustrated in FIG. 16. FIG. 16 is a hardware configuration diagram illustrating an example of the computer 1000 that implements the functions of the signal processing unit 123 and the information processing unit 103. The computer 1000 includes a CPU 1100, a RAM 1200, a ROM (Read Only Memory) 1300, a HDD (Hard Disk Drive) 1400, a communication interface 1500, and an input/output interface 1600. The units of the computer 1000 are connected by a bus 1050.

The CPU 1100 operates based on programs stored in the ROM 1300 or the HDD 1400 and controls the units. For example, the CPU 1100 develops the programs stored in the ROM 1300 or the HDD 1400 in the RAM 1200 and executes processing corresponding to various programs.

The ROM 1300 stores a boot program such as a BIOS (Basic Input Output System) to be executed by the CPU 1100 at a start time of the computer 1000, a program depending on hardware of the computer 1000, and the like.

The HDD 1400 is a computer-readable recording medium that non-transiently records a program to be executed by the CPU 1100, data to be used by such a program, and the like. Specifically, the HDD 1400 is a recording medium that records a program for realizing the operations according to the present disclosure, which is an example of program data 1450.

The communication interface 1500 is an interface for the computer 1000 to be connected to an external network 1550 (for example, the Internet). For example, the CPU 1100 receives data from other equipment and transmits data generated by the CPU 1100 to the other equipment via the communication interface 1500.

The input/output interface 1600 is an interface for connecting the input/output device 1650 and the computer 1000. For example, the CPU 1100 receives data from an input device such as a keyboard or a mouse via the input/output interface 1600. The CPU 1100 transmits data to an output device such as a display, a speaker, or a printer via the input/output interface 1600. The input/output interface 1600 may function as a media interface that reads a program or the like recorded in a predetermined recording medium (a medium). The medium is, for example, an optical recording medium such as a DVD (Digital Versatile Disc) or a PD (Phase change rewritable Disk), a magneto-optical recording medium such as an MO (Magneto-Optical disk), a tape medium, a magnetic recording medium, or a semiconductor memory.

For example, when the computer 1000 functions as the signal processing unit 123 and the information processing unit 103 according to the embodiment explained above, the CPU 1100 of the computer 1000 implements the functions of the signal processing unit 123 and the information processing unit 103 by executing a program loaded on the RAM 1200. A program and the like according to the present disclosure are stored in the HDD 1400. Note that the CPU 1100 reads the program data 1450 from the HDD 1400 and executes the program data 1450. However, as another example, the CPU 1100 may acquire these programs from another device via the external network 1550.

### 4. Modifications

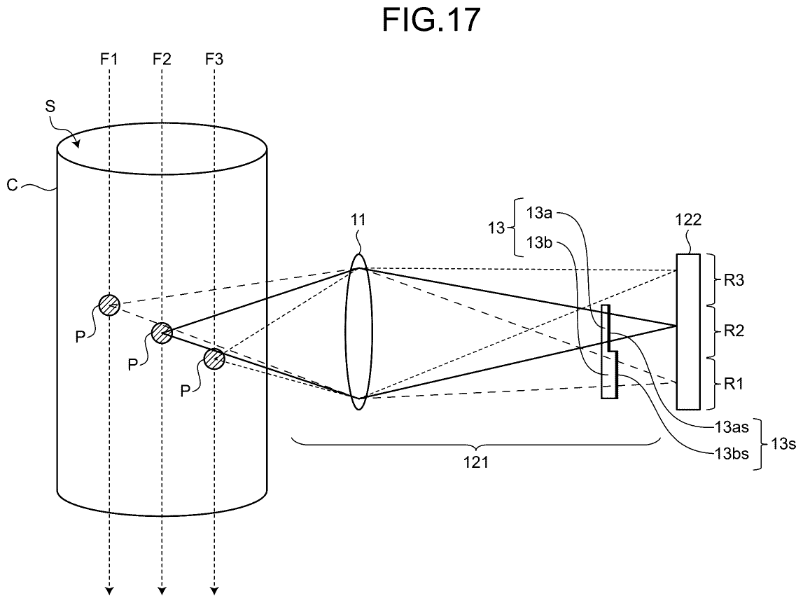

As explained above, a plurality of images having different focal positions are acquired by using the objective lens 11 and the optical path length adjustment element 13 in combination. In this case, spherical aberration can occur at each of focal positions. In an embodiment, the optical path length adjustment element 13 may be configured to correct the spherical aberration. This is explained with reference to FIG. 17 to FIG. 19 as well.

FIG. 17 is a diagram illustrating a modification. Note that the optical path length adjustment element 13 illustrated in FIG. 17 is disposed such that the surfaces respectively facing the objective lens 11 and the imaging unit 122 are reversed compared with FIG. 7 and the like referred to above. However, such an aspect can also be one of embodiments.

As explained above, the optical path length adjustment element 13 includes the first portion 13a and the second portion 13b. The light from the microparticles P on the path F1 passes through the objective lens 11 and the second portion 13b of the optical path length adjustment element 13 and forms an image in the region R1 of the imaging unit 122. The light from the microparticles P on the path F2 passes through the objective lens 11 and the first portion 13a of the optical path length adjustment element 13 and forms an image in the region R2 of the imaging unit 122. The light from the microparticles P on the path F3 passes through the objective lens 11 and, on the other hand, forms an image in the region R3 of the imaging unit 122 without passing through the optical path length adjustment element 13.

Here, it is assumed that the focal position of the objective lens 11 is adjusted to the path F3. In this case, spherical aberration correction for the light from the microparticles P on the path F3 is unnecessary or less necessary. The spherical aberration of the light from the microparticles P on the path F1 and the spherical aberration of the light from the microparticles P on the path F2 are corrected by the optical path length adjustment element 13. Specifically, the optical path length adjustment element 13 has a surface shape for correcting the spherical aberration.

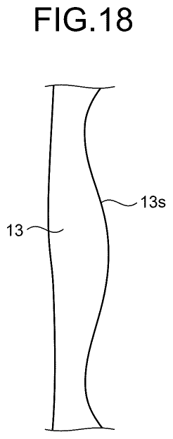

The surface of the optical path length adjustment element 13, more specifically, in this example, a surface facing the imaging unit 122 is referred to and illustrated as surface 13s. In the surface 13s of the optical path length adjustment element 13, the surface of the first portion 13a is referred to and illustrated as surface 13as. The surface of the second portion 13b is referred to and illustrated as surface 13bs. The surface 13s is explained with reference to FIG. 18 as well.

FIG. 18 is a diagram illustrating an example of a surface shape of the optical path length adjustment element. In this example, the optical path length adjustment element 13 has a spherical aberration correction shape similar to the shape of a Schmitt correction plate. The surface 13s of the optical path length adjustment element 13 is not a flat surface but includes a gentle curved surface. This curved surface can also be referred to as curved surface formed to correct spherical aberration or curved surface that provides a spherical correction shape to the optical path length adjustment element 13. Note that the shape appearing in FIG. 18 is schematic A more specific shape is designed as appropriate.

Returning to FIG. 17, for example, each of the surface 13as of the first portion 13a and the surface 13bs of the second portion 13b of the optical path length adjustment element 13 has a shape for correcting spherical aberration of light corresponding thereto. Specifically, the surface 13as of the first portion 13a has a shape for correcting spherical aberration of light from the microparticles P on the path F2. The light, the spherical aberration of which is corrected, forms an image in the region R2 of the imaging unit 122 and a clear image is obtained. The surface 13bs of the second portion 13b has a shape for correcting spherical aberration of light from the microparticles P on the path F1. The light, the spherical aberration of which is corrected, forms an image in the region R3 of the imaging unit 122 and a clear image is obtained. For the light from the microparticles P on the path F3, spherical aberration does not have to be corrected as explained above. The light forms an image in the region R3 of the imaging unit 122 without passing through the optical path length adjustment element 13 and a clear image is obtained. Therefore, the clear image with reduced spherical aberration can be obtained in all of the region R1 to the region R3 of the imaging unit 122.

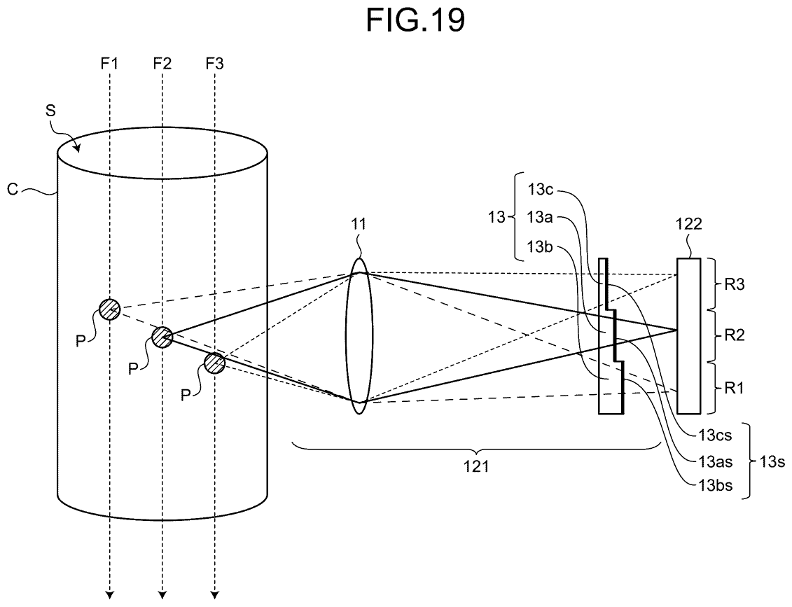

FIG. 19 is a diagram illustrating another modification. The optical path length adjustment element 13 further includes a third portion 13c. In this example, the third portion 13c is a portion opposite to the second portion 13b across the first portion 13a and has thickness smaller than the thickness of the first portion 13a. The light from the microparticle P on the path F3 passes through the objective lens 11 and the third portion 13c of the optical path length adjustment element 13 and forms an image. A position of this image formation is the position of the region R3 of the imaging unit 122.

The surface of the third portion 13c is referred to and illustrated as surface 13cs. The surface 13cs of the third portion 13c has a shape for correcting spherical aberration of light from the microparticles P on the path F3. An example of the shape is as explained above with reference to FIG. 18. The light, the spherical aberration of which is corrected, forms an image in the region R1 of the imaging unit 122 and a clear image is obtained.

With the optical path length adjustment element 13 illustrated in FIG. 19, for example, even if the focal position of the objective lens 11 is not adjusted to all of the path F1, the path F2, and the path F3, the spherical aberration of the light from the microparticles P on the paths can be corrected.

Although the embodiment of the present disclosure is explained above, the technical scope of the present disclosure is not limited to the embodiment explained above per se. Various changes are possible without departing from the gist of the present disclosure. Components in different embodiments and modifications may be combined as appropriate.

The effects in the embodiments described in this specification are only illustrations and are not limited. Other effects may be present.

Note that the present technique can also take the following configurations.
(1) A biological sample analysis system comprising:
   a detection optical system, a focal point of which is set at a predetermined position in a container;
   an imaging unit, a light receiving surface of which is located at an image forming position of an image of light transmitted through the detection optical system; and
   an optical path length adjustment element disposed on an optical path between the detection optical system and the imaging unit and in a part within an angle of view of the imaging unit.
(2) The biological sample analysis system according to (1), wherein
   an image of light transmitted through the detection optical system and the optical path length adjustment element is formed at a position different from an image forming position on the light receiving surface of an image of light transmitted through the detection optical system and not transmitted through the optical path length adjustment element.
(3) The biological sample analysis system according to (1) or (2), wherein
   the optical path length adjustment element includes a plurality of regions having stepwise different thicknesses along the optical path of the light.
(4) The biological sample analysis system according to any one of (1) to (3), wherein
   a surface of the optical path length adjustment element includes a curved surface.
(5) The biological sample analysis system according to any one of (1) to (4), wherein
   a distance between a focal position in the container of the detection optical system with respect to the light that is transmitted through the detection optical system to form an image on the light receiving surface without being transmitted through the optical path length adjustment element and a focal position in the container of the detection optical system with respect to the light that is transmitted through the detection optical system and the optical path length adjustment element to form an image on the light receiving surface is shorter than a diameter of particles present in the container.
(6) The biological sample analysis system according to any one of (1) to (5), wherein
   the detection optical system includes an objective lens, the focal point of which is set at the predetermined position in the container.
(7) The biological sample analysis system according to (6), wherein
   the detection optical system further includes an image forming lens that forms an image of the light transmitted through the objective lens on the light receiving surface of the imaging unit.
(8) The biological sample analysis system according to (7), wherein
   the detection optical system further includes a spectroscopic element that is disposed between the objective lens and the image forming lens and disperses light transmitted through the objective lens.
(9) The biological sample analysis system according to any one of (1) to (8), wherein
   the imaging unit includes any one of an image sensor, an EVS (Event-based Vision Sensor), and an SPAD (Single-Photon Avalanche Diode) sensor.
(10) The biological sample analysis system according to (9), wherein
   the imaging unit detects the image of the light with a TDI (Time Delay Integration) scheme.
(11) The biological sample analysis system according to any one of (1) to (10), wherein
   the imaging unit detects the image of the light from particles moving in the container and outputs image data.
(12) The biological sample analysis system according to (11), further comprising
   an evaluation unit that evaluates a focus state of the image data output from the imaging unit.
(13) The biological sample analysis system according to (12), wherein
   the imaging unit outputs first image data including an image of light formed on the light receiving surface without passing through the optical path length adjustment element and second image data including an image of light formed on the light receiving surface after passing through the optical path length adjustment element, the images being images of light from the same particles, and
   the evaluation unit evaluates a focus state of each of the first image data and the second image data and selects image data with higher evaluation.
(14) The biological sample analysis system according to (13), wherein
   the evaluation unit evaluates the focus state of each of the first image data and the second image data by evaluating a region corresponding to a same region of the particles in each of the first image data and the second image data.
(15) The biological sample analysis system according to any one of (12) to (14), wherein
   the evaluation unit evaluates the focus state of the image data based on at least one of an integrated value of a luminance difference between adjacent pixels in an entire or a part of a region of the image data, an intensity value of a high-frequency component in the entire or the part of the region of the image data, and an external dimension of an image of the particles in the image data.
(16) The biological sample analysis system according to any one of (11) to (15), wherein
   the particles are bioparticles.
(17) The biological sample analysis system according to (16), wherein
   the container is a channel through which a liquid sample containing the bioparticles flows.
(18) A biological sample analysis method executed in a biological sample analysis system including: a detection optical system, a focal point of which is set at a predetermined position in a container; an imaging unit, a light receiving surface of which is located at an image forming position of an image of light transmitted through the detection optical system; and an optical path length adjustment element disposed on an optical path between the detection optical system and the imaging unit and in a part within an angle of view of the imaging unit, the imaging unit detecting the image of the light from particles moving in the container and outputting image data, the biological sample analysis method comprising
   evaluating a focus state of the image data output from the imaging unit.

### REFERENCE SIGNS LIST

11 OBJECTIVE LENS
12 MOVING MECHANISM
13 OPTICAL PATH LENGTH ADJUSTMENT ELEMENT
13a FIRST PORTION
13b SECOND PORTION
14 IMAGE FORMING LENS
15 SPECTROSCOPIC ELEMENT
100 PARTICLE ANALYSIS SYSTEM
101 LIGHT IRRADIATION UNIT
102 DETECTION UNIT
103 INFORMATION PROCESSING UNIT
104 SORTING UNIT
105 NUMBER-OF-PARTICLES MEASUREMENT UNIT
106 WELL PLATE
111 LIGHT SOURCE UNIT FOR DETECTION
112 LIGHT GUIDE OPTICAL SYSTEM
121 DETECTION OPTICAL SYSTEM
122, 122-1 to 122-3 IMAGING UNIT
122a IMAGE SENSOR
122b EVS
123 SIGNAL PROCESSING UNIT
124 SPEED MEASUREMENT UNIT
A1 to A3, A11 to A13 IMAGE FORMING POSITION
C CHANNEL
F1 to F3 PATH
P BIOPARTICLE
S BIOLOGICAL SAMPLE

What is claimed is:

1. A biological sample analysis system comprising:
a detection optical system, a focal point of which is set at a predetermined position in a container;
an imaging unit, a light receiving surface of which is located at an image forming position of an image of light transmitted through the detection optical system; and
an optical path length adjustment element disposed on an optical path between the detection optical system and the imaging unit and in a part within an angle of view of the imaging unit.

2. The biological sample analysis system according to claim 1, wherein
an image of light transmitted through the detection optical system and the optical path length adjustment element is formed at a position different from an image forming position on the light receiving surface of an image of light transmitted through the detection optical system and not transmitted through the optical path length adjustment element.

3. The biological sample analysis system according to claim 1, wherein
the optical path length adjustment element includes a plurality of regions having stepwise different thicknesses along the optical path of the light.

4. The biological sample analysis system according to claim 1, wherein a surface of the optical path length adjustment element includes a curved surface.

5. The biological sample analysis system according to claim 1, wherein
a distance between a focal position in the container of the detection optical system with respect to the light that is transmitted through the detection optical system to form an image on the light receiving surface without being transmitted through the optical path length adjustment element and a focal position in the container of the detection optical system with respect to the light that is transmitted through the detection optical system and the optical path length adjustment element to form an image on the light receiving surface is shorter than a diameter of particles present in the container.

6. The biological sample analysis system according to claim 1, wherein
the detection optical system includes an objective lens, the focal point of which is set at the predetermined position in the container.

7. The biological sample analysis system according to claim 6, wherein
the detection optical system further includes an image forming lens that forms an image of the light transmitted through the objective lens on the light receiving surface of the imaging unit.

8. The biological sample analysis system according to claim 7, wherein
the detection optical system further includes a spectroscopic element that is disposed between the objective lens and the image forming lens and disperses light transmitted through the objective lens.

9. The biological sample analysis system according to claim 1, wherein
the imaging unit includes any one of an image sensor, an EVS (Event-based Vision Sensor), and an SPAD (Single-Photon Avalanche Diode) sensor.

10. The biological sample analysis system according to claim 9, wherein
the imaging unit detects the image of the light with a TDI (Time Delay Integration) scheme.

11. The biological sample analysis system according to claim 1, wherein
the imaging unit detects the image of the light from particles moving in the container and outputs image data.

12. The biological sample analysis system according to claim 11, further comprising
an evaluation unit that evaluates a focus state of the image data output from the imaging unit.

13. The biological sample analysis system according to claim 12, wherein
the imaging unit outputs first image data including an image of light formed on the light receiving surface without passing through the optical path length adjustment element and second image data including an image of light formed on the light receiving surface after passing through the optical path length adjustment element, the images being images of light from the same particles, and
the evaluation unit evaluates a focus state of each of the first image data and the second image data and selects image data with higher evaluation.

14. The biological sample analysis system according to claim 13, wherein
the evaluation unit evaluates the focus state of each of the first image data and the second image data by evaluating a region corresponding to a same region of the particles in each of the first image data and the second image data.

15. The biological sample analysis system according to claim 12, wherein
the evaluation unit evaluates the focus state of the image data based on at least one of an integrated value of a luminance difference between adjacent pixels in an entire or a part of a region of the image data, an intensity value of a high-frequency component in the entire or the part of the region of the image data, and an external dimension of an image of the particles in the image data.

16. The biological sample analysis system according to claim 11, wherein
the particles are bioparticles.

17. The biological sample analysis system according to claim 16, wherein
the container is a channel through which a liquid sample containing the bioparticles flows.

18. A biological sample analysis method executed in a biological sample analysis system including: a detection optical system, a focal point of which is set at a predetermined position in a container; an imaging unit, a light receiving surface of which is located at an image forming position of an image of light transmitted through the detection optical system;
and an optical path length adjustment element disposed on an optical path between the detection optical system and the imaging unit and in a part within an angle of view of the imaging unit, the imaging unit detecting the image of the light from particles moving in the container and outputting image data, the biological sample analysis method comprising
evaluating a focus state of the image data output from the imaging unit.
